(19) United States
(12) Patent Application Publication
(10) Pub. No.: US 2007/0014743 A1
(43) Pub. Date: Jan. 18, 2007

(54) Subunguicide, and method for treating onychomycosis

(76) Inventor: Jay E. Birnbaum, Montville, NJ (US)

Correspondence Address: BRADLEY N. RUBEN, PC, 463 FIRST ST, SUITE 5A, HOBOKEN, NJ, 07030, US

(21) Appl. No.: 11/523,508

(22) Filed: Sep. 19, 2006

Related U.S. Application Data
(63) Continuation-in-part of application No. 10/671,307, filed on Sep. 25, 2003, now Pat. No. 7,135,194.
(60) Provisional application No. 60/414,012, filed on Sep. 27, 2002.

(51) Int. Cl. A61K 8/49

(52) U.S. Cl. 424/61

(57) ABSTRACT

Onychomycosis is a fungal infection of the nail bed and is difficult to treat topically because penetration of the nail plate is difficult, and systemic antifungal treatments are prone to side effects and drug interactions. The present invention treats onychomycosis by applying an antifungal composition to the nail bed directly using a solid, semi-solid, or flowable carrier. The carrier can be in the form of a semi-solid into which the user digs and scrapes the nail, a solid carrier can be inserted directly under the nail in contact with the nail bed, or a flowable composition can be injected in contact with the nail bed.

### RELATED APPLICATIONS

This application is a continuation-in-part of application Ser. No. 10/671,307, filed Sep. 25, 2003, which is based on provisional application Ser. No. 60/414,012, filed Sep. 27, 2002, the disclosures of which are incorporated herein by reference.

### BACKGROUND OF THE INVENTION

1. Field of the Invention.

This invention relates to articles of manufacture and to methods for treating subungual (under the nail) infections, especially fungal infections (onychomycosis).

2. The State of the Art.

Fungi are eukaryotic cells that may reproduce sexually or asexually and may be biphasic, with one form in nature and a different form in the infected host. Fungal diseases are referred to as mycoses.

A fungal infection of the nails, commonly referred to as onychomycosis, is most frequently caused by dermatophytes but also can be caused by molds and Candida. Mixed infections also occur. Onychomycosis includes dermatophyte infection of the nail by any fungus, including yeast or molds. Thus, for example, onychomycosis serves as a reservoir for dermatophytes and contributes to treatment failure and recurrence of tinea pedis. Most common causes of tinea unguium are Trichophyton rubrum (most frequent), T. mentagrophytes, and Epidermophyton floccusum. These are dermatophytes (fungi that infect hair, skin, and nails) and feed on keratinized (nail) tissue. The nail infections they cause are normally confined to the nail bed and nail plate, but occasionally spread to the surrounding skin. Another type of onychomycosis is caused by yeast (e.g., Candida albicans or Candida parapsilosis). These infections are less common and produce similar symptoms.

The majority of known antifungal agents fall into one of three main groups. One major group includes polyene derivatives, including amphotericin B and the structurally related compounds nystatin and pimaricin, which are only administered intravenously. These are broad-spectrum antifungals that bind to ergosterol, a component of fungal cell membranes, and thereby disrupt the membranes, leading to cell death. Amphotericin B is usually effective for systemic mycoses, but its administration is limited by toxic effects that include fever and kidney damage, and other accompanying side effects such as anemia, low blood pressure, headache, nausea, vomiting and phlebitis. The unrelated antifungal agent flucytosine (5-fluorocytosine, a diazine), an orally absorbed drug, is frequently used as an adjunct to amphotericin B treatment for some forms of candidiasis and cryptococcal meningitis. Its adverse effects include bone marrow depression with leukopenia and thrombocytopenia.

A second major group of antifungal agents includes azole derivatives which impair synthesis of ergosterol and lead to accumulation of metabolites that disrupt the function of fungal membrane-bound enzyme systems (e.g., CYP-26, i.e., cytochrome P450) and inhibit fungal growth. Significant inhibition of mammalian CYP-26 results in important drug interactions. This group of agents includes ketoconazole (U.S. Pat. Nos. 4,144,346 and 4,223,036), fluconazole (U.S. Pat. No. 4,404,216), itraconazole (U.S. Pat. No. 4,267,179), liarozole, irtemazol, clotrimazole, miconazole, econazole, butoconazole, oxiconazole, sulconazole, and terconazole. U.S. Pat. No. 6,277,873 describes substituted thiazole, thiadiazole, and oxadiazole antifungals.

Antifungal azoles are fungistatic, not fungicidal, which has resulted in azole resistant fungi, that is, fungi strains and isolates which are resistant to treatment with Fluconazole and other known antifungal agents (New Engl. J. Med., 1944, 330: 263-272.) The small concentration of topical antifungal agents penetrating the nail through to the bed might contribute to the development of fungi resistant to therapeutic agents.

A third major group of antifungal agents includes the fungicidal allylamines such as naftifine (Naftin), terbinafine (EP 24,587-A1; Lamisil), and the benzylamine butenafine (Mentax).

Yet another antifungal is the commonly used thiocarbonate tolnaftate. Like the allylamines and azoles, tolnaftate blocks synthesis of ergosterol.

Various other types of antifungal agents are known. Griseoflulvin is a fungistatic agent which is administered orally for fungal infections of skin, hair or nails that do not respond to topical treatment. U.S. Pat. No. 6,221,903 describes the use of Amiodarone, a Class III antiarrhythnic drug (Amiodarone in Physicians GenRx, 1996, BeDell, et. al, eds., Mosby-Year Book, Inc., St. Louis, Mo.; Amiodarone in Drug Information for the HealthCare Profession, 1997, USP DI, Twinbrook Parkway, Md.; pp. 80-83), as an antifungal agent. Still other antifungal agents include ciclopirox, sulbentine, and morpholines, e.g., amorolfine, and the related morpholines disclosed in U.S. Pat. No. 5,120,530, and the 1-hydroxy-2-pyridone compounds disclosed in U.S. Pat. No. 4,957,730.

It has also been known to combine antifungal agents with anti-inflammatory agents. The steroidal anti-inflammatory agent may be selected from among any of the known steroidal anti-inflammatory agents, including, for example, any of those disclosed in The Merck Index or in U.S. Pat. Nos. 5,002,938, 5,110,809, and 5,219,877. Examples of steroidal anti-inflammatory agents useful in combination with antifungals can include 21-acetoxypregnenolone, alclometasone or its dipropionate salt, algestone, amcinonide, beclomethasone or its dipropionate salt, betamethasone and salts thereof, including, for example, betamethasone benzoate, betamethasone dipropionate, betamethasone sodium phosphate, betamethasone sodium phosphate and acetate, and betamethasone valerate; clobetasol or its propionate salt, clocortolone pivalate, hydrocortisone and salts thereof, including, for example, hydrocortisone acetate, hydrocortisone butyrate, hydrocortisone cypionate, hydrocortisone phosphate, hydrocortisone sodium phosphate, hydrocortisone sodium succinate, hydrocortisone tebutate and hydrocortisone valerate; cortisone acetate, desonide, desoximetasone, dexamethasone and salts thereof, for example, acetate and sodium phosphate; diflorasone diacetate, fludrocortisone acetate, flunisolide, fluocinolone acetonide, fluocinonide, fluorometholone, flurandrenolide, halcinonide, medrysone, methylprednisolone and salts thereof, e.g., acetate, sodium succinate; mometasone furoate, paramethasone acetate, prednisolone and salts thereof, e.g., acetate, diethylaminoacetate, sodium phosphate, sodium succinate, tebutate, trimethylacetate; prednisone, triamcinolone and derivatives thereof, e.g., acetonide, benetonide, diacetate, hexacetonide. Other glucocorticoid steroids reported in the literature, including The Merck Index, or otherwise approved by the local drug regulatory agency, e.g., Food and Drug Administration, may also be used. Preferred steroidal anti-inflammatory agents usually include clobetasol and its salts, e.g., propionate salt; betamethasone and its salts, hydrocortisone and its salts, and triamcinolone and its salts, although as new steroidal anti-inflammatories are developed and reviewed, preferences may change. The anti-inflammatory agent will usually be present in a topical composition in combination with an antifungal in an amount within the range of 0.01 to about 5 percent, preferably from about 0.1 to 2 percent, based on the total weight of the composition.

Thus, various types of antifungal agents and their combination with steroidal anti-inflammatory agents are known.

In spite of the wide varieties of anti-fungal and fungistatic agents, and their use in combination with other active ingredients, onychomycosis is difficult to treat. Since most onychomycosis (i.e., the distal subungual form) is a disease of the nail bed underlying the nail plate, the condition is best treated systemically (from the inside) because topical access to the nail bed is not present. Accordingly, most onychomycosis is treated using oral medications such as terbinafine (Lamisil) and itraconazole (Sporonox). The nail grows slowly, and so systemic (oral) medicines require several months for elimination of the infection and regrowth of new nail. These drugs may also produce serious side effects, and they may interact with other medications. Accordingly, systemic medications for treating onychomycosis are unacceptable for many patients. For those patients, the only available route of administration is topical.

Nail lacquers for the treatment of onychomycoses and similar fungal infections affecting nails (toe nails and/or finger nails) of humans, in particular, or other animals, are known. Representative examples are described in the patent literature, such as the following U.S. Pat. No.: 4,957,730 (1-hydroxy-2-pyridone in water-insoluble film-former); U.S. Pat. No. 5,120,530 (amorolfine in quaternary ammonium acrylic copolymer); U.S. Pat. No. 5,264,206 (tioconazole, econazole, oxiconazole, miconazole, tolnaftate, naftifine hydrochloride, in water-insoluble film-former); U.S. Pat. No. 5,346,692 (with urea and dibutyl phthalate plasticizer); U.S. Pat. No. 5,487,776 (griseofulvin as colloidal suspension). U.S. Pat. No. 6,224,887, teaches a nail lacquer for onychomycosis with combination of antifungal and a certain penetration-enhancing medium carbon chain dioxane or acetal. PENLAC brand ciclopirox is the only FDA-approved topical treatment approved in the United States for onychomycosis.

Other U.S. Pat. Nos. which relate to antifungal products include, for example, U.S. Pat. No. 4,636,520 (combination of imidazole and pyrrolnitrin); U.S. Pat. No. 5,002,938 (gel, combination of imidazole and 17-ester corticosteroid anti-inflammatory agent); U.S. Pat. No. 5,110,809 (antifungal gel plus steroid); U.S. Pat. No. 5,219,877 (gel product with imidazole antifungal optionally with steroidal anti-inflammatory, in a vehicle system that includes lauryl alcohol); U.S. Pat. No. 5,391,367 (aqueous alcoholic gel with tioconazole); U.S. Pat. No. 5,464,610 (salicylic acid plaster); and U.S. Pat. No. 5,696,105 (mometasone furoate).

U.S. Pat. No. 6,207,142 describes antifungal shampoos.

U.S. Pat. no. 5,894,020, discloses an antifungal bar soap for treating tinea pedis.

Anatomically, the “nail” that is seen is technically the nail plate. As shown in FIG. 1, a perspective cross-sectional view of the proximal part of a digit 101, soft tissue 103 overlies the distal phalanx 105 (not shown in other figures), and the majority and distal end of the nail plate 107 overlies the most proximal part of the nail bed 109; the root 111 of the nail plate overlies the nail matrix 113 from which the nail grows. The eponychium 123 (the cuticle) forms a seal between the skin 125 and the proximal end of the nail plate. At the distal end, between the nail plate and the skin, is the hyponychium 127, which is a physical barrier sealing the distal margin of the nail bed where it is coextensive with the nail plate. The nail plate presents a considerable barrier to dorsal (orthogonal) penetration and hence limits access to the nail bed for drugs intended for the nail bed and applied topically to the nail plate. Current topical therapies have such low penetration through the nail plate that they have a very low efficacy (less than 10% even after prolonged application). These therapies do not appear to exhibit characteristic concentration-response or time-response relationships. This suggests that in the small percentage of people in whom these topical treatments are effective, efficacy may not be related to penetration through the nail. Materials such as urea increase the penetration of the medication through the nail plate, but such materials alter the nail and disrupt its integrity.

### SUMMARY AND OBJECTS OF THE INVENTION

In light of the forgoing, it would benefit the treatment of onychomycosis to administer an antifungal agent in closer proximity to the nail bed, and to decrease the barriers to access to the nail bed to treat the condition. As mentioned above, there is a small population that is helped by topical treatment, in spite of low penetration through the nail. Successful treatment in those people suggests that administration in contact with the nail bed and/or cutting/manipulation of the nail may have significantly contributed to a favorable outcome. Accordingly, a more reasonable approach to topical treatment, especially in cases where only the distal half (or less) of the nail bed is involved, is with subungual treatment.

Another object of this invention is to provide a novel solid or semisolid subunguicide.

Accordingly, in one embodiment this invention provides a method for treating onychomycosis by administering an antifungal subungually between the nail plate and the hyponychium.

In order to practice this treatment without invasive procedures or compromising the nail plate, another embodiment of this invention provides a solid or semisolid material that is placed in contact with the nail bed by introduction between the nail plate and the hyponychium.

### BRIEF DESCRIPTION OF THE DRAWINGS

FIG. 1 is an idealized section through the distal portion of a digit depicting the anatomy.

FIGS. 2A through 2D depict the administration of a subunguicide by different devices.

FIGS. 3A through 3C are depictions over time, and with repeated administration, of the migration of the administered subunguicide along the nail bed.

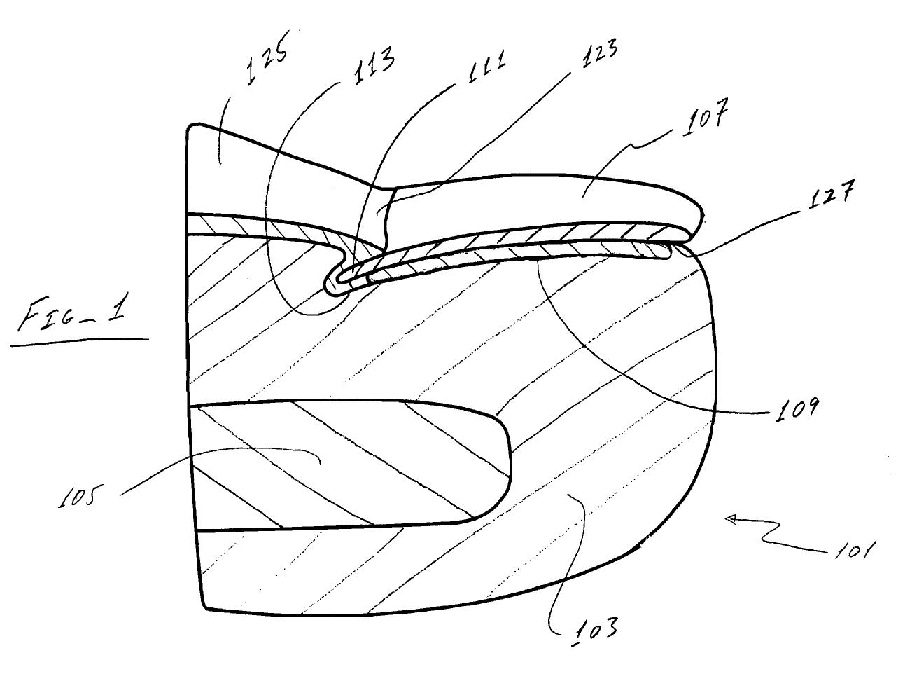

### DESCRIPTION OF SPECIFIC EMBODIMENTS

Antifungal agents and various compositions containing the same are described in the Background section, and the patents and literature references mentioned therein are incorporated herein by reference.

By this invention, a solid or semisolid material having a fungicidal or fungistatic agent is placed in contact with the nail bed and preferably forced under the nail plate. Placing the antifungal in such close proximity provides the preferred direct contact between the infected area and the therapeutic agent. In addition, having a small reservoir of antifungal agent facilitates diffusion of antifungal proximally along the nail bed. Localized therapy in this fashion avoids problems with the present therapies based on systemic administration because systemic administration can increase the possibility of any side effects of the antifungal being manifest as well as potential interactions with concomitant medications.

As noted in the Background section, the manufacture of bar soaps, shampoos, and gels, having antifungal agents are known. It is also well-known how to formulate caulks, pastes, and cream (such as topical delivery creams and dentifrices) and semisolids (such as deodorant and antiperspirant/deodorant sticks) for cosmetic and pharmaceutical applications.

As one example, bar soap is commercially available having a desired antifungal agent (such as zinc pyrithione), or combination of antifungal agents, and optionally other active agents (e.g., an anti-inflammatory) and/or inactive agents (e.g., colorants, fragrances, conditioners, humectants). Such a product is used according to this invention by having the patient scratch the bar with the infected nail(s) effective to cause the soap shavings to reside under the nail and in contact with the nail bed. The antifungal agent can be suspended in any sort of soft solid or semisolid material, and/or it can be dispersed as solid particles. The physical properties of the solid or semisolid multidose bar, such as hardness (within the range of hardnesses that soaps can be manufactured; hardness being measured on a scale such as the Mohs hardness scale, and softer than a human nail) can be adjusted to facilitate the penetration of the soap under the nail. The bar matrix carrier for the antifungal can be a material that softens or even one that melts slowly at the surface temperature under the nail. Semisolid carriers (such as used for deodorants and antiperspirants, and cosmetics) can be formulated as desired to deliver the antifungal by having the patient scratch the substance (whether or not in the geometry of a bar).

A caulk or paste, or a gel, can be forced under the nail. The rheology of such a material can be adjusted to facilitate its being forced subungually between the nail plate and the nail bed when dispensed as it experiences different shear rates when under the nail confines than when flowing through a dispenser outlet or forced in with a spatula.

In a similar embodiment, a more flowable composition, such as a cream, ointment, solution, or suspension can be placed under the nail by means of an applicator inserted between the nail bed and the nail plate. Such an applicator can be a hypodermic needle or similar device for injecting by pressure, a cannula through which a sponge or other porous carrier is inserted, or other small tube through which the antifungal may be carried.

In an analogous manner, a small strip or pellet can be placed under the nail in contact with the nail bed, or force between the nail plate and the nail bed. The strip or pellet can be a polymer coated with an antifungal, or a hard sponge or porous polymer coated and/or infiltrated with an antifungal, or any other excipient sufficiently hard to be placed under the nail, and preferably to be forced at least partially between the nail plate and the nail bed.

For those administration devices that can be forced between the nail plate and the nail bed, the addition of a topical anesthetic and/or short-acting vasoconstrictor (to minimize bleeding) may be desirable.

Thus, while the prior art attemts to treat the condition systemically, through the nail plate, or “transungually”, the present invention accesses the nail bed by administration between the hyponychium and the nail plate to so that the drug rests in contact with the nail bed. During onychomycosis, onycholysis, or lifting of the nail plate from the nail bed, is a frequent occurrence. This onycholysis occurs because of a rapid cell turnover of the nail bed epithelium, caused by the inflammatory response to the onychomycotic fungal infection. It can be beneficial to the subungual delivery methods and compositions disclosed herein by providing some space between the nail plate and the hyponychium, allowing the medication to be forced past (over) the hyponychium and administered to the distal part of the nail bed, which where it will diffuse or migrate proximally and laterally.

Yet another method for delivering the medication is with a jet injector (high pressure injection). Such devices are typically used for insulin (in diabetes patients) and for innoculations, and force the liquid substance to be delivered through the skin. For the present invention, a jet injector, preferably adapted to provide a nozzle suitable for contact with the hyponychium, can be used to administer the medication directly to the nail bed.

After administration of the medication, the subungual area can be occluded, such as with a small bandage (physical and/or a film-forming substance). A finger cot or a glove (for a hand or foot (i.e., a sock with separate extensions for each toe)) can be used to occlude the end of the digit, or multiple digits.

The amount of the active antifungal agent or mixture of such agents in the composition will depend on such factors as its structure and antimicrobial activity, release rate from the gel/paste/semisolid/solid carrier, and diffusion characteristics. Generally, the effective amount of the anitfungal agent in any given dose will be several to several tens to hundreds of times greater than the Minimal Inhibitory Concentration (MIC). Typically, amounts of active antifungal agent in the range of from about 0.5 to 20 percent by weight, preferably from about 1 to 10 percent, by weight, of the total composition.

The present composition and method can also use a keratolytic agent to facilitate diffusion or migration of the medication through the subungal debris, caused during the above-described onycholysis. Suitable keratolytic agents include urea (5-40%), salicylic acid (5-40%), DMSO, sulfur, and other known compounds. Acid and/or enzymatic keratolytics can be used. The acids include the alpha-hydroxy acids (such as lactic acid), beta-hydroxy acids (such as salicylic acid), and their derivatives such as keto-hydroxy acids, including the root moieties glycolic, lactic, pyruvic, and citric. In addition, such derivatives can include salts, such as ammonium lactate (commercially available as LacHydrin). Examples of enzymatic exfoliants useful in the compositions and methods of the invention include, but are not limited to, papain, from papaya, and bromalein, from pineapple. Examples of acidic exfoliants include, but are not limited to a mono- or poly-hydroxy acid, tannic acid, or a mixture thereof, or a pharmaceutically acceptable salt or ester thereof. One of ordinary skill in the art will be readily able to select and prepare suitable mono- or poly-hydroxy acids for use in the composition of the invention, for example, alkyl hydroxycarboxylic acids, aralkyl and aryl hydroxycarboxylic acids, polyhydroxy-carboxylic acids, and hydroxy-polycarboxylic acids. One of ordinary skill in the art would typically select one or more of the following mono- or poly-hydroxy acids: 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid; 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutaned-ioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate, ethyl benzoyl formate and propyl benzoyl formate; 4-hydroxybenzoyl formic acid and esters thereof; 4-hydroxyphenyl pyruvic acid and esters thereof, and 2-hydroxyphenyl pyruvic acid and esters thereof.

As mentioned above, acceptable salts of the foregoing acids can be used as keratolytic agents. Examples of suitable inorganic metallic bases for salts formation with the acid compounds of the invention include, but are not limited to, ammonium, aluminum, calcium, lithium, magnesium, potassium, sodium, and zinc. Appropriate organic bases may be selected, for example, from N,N-dibenzylethylenediamine, chloroprocaine, choline, diethanolamine, ethylenediamine, meglumaine (N-methylglucamine), and procaine. It should be understood that one or more derivatives of the above acidic component, such as esters or lactones thereof, are also suitably used. One of ordinary skill in the art will also understand that various hydroxy acids described in U.S. Pat. Nos. 5,547,988 and 5,422,370, the disclosures of which are incorporated herein by reference, are also suitable for use in the compositions and methods of the invention. The acidic component is present in the composition and methods in an amount sufficient to exfoliate, i.e., remove dead or dying cells, from at least a portion of the nail bed. The acidic component is typically present in an amount from about 0.1 to 12 weight percent, preferably about 1 to 11 weight percent, more preferably from about 4 to 10 weight percent of the composition. For example, the acidic component may be from about 0.1 to 3 weight percent citric acid in combination with up to about 2 weight percent salicylic acid.

The instant composition is preferably applied between twice daily and once weekly, more preferably between once daily and once every three days. It is also preferred that the doses be self-administered.

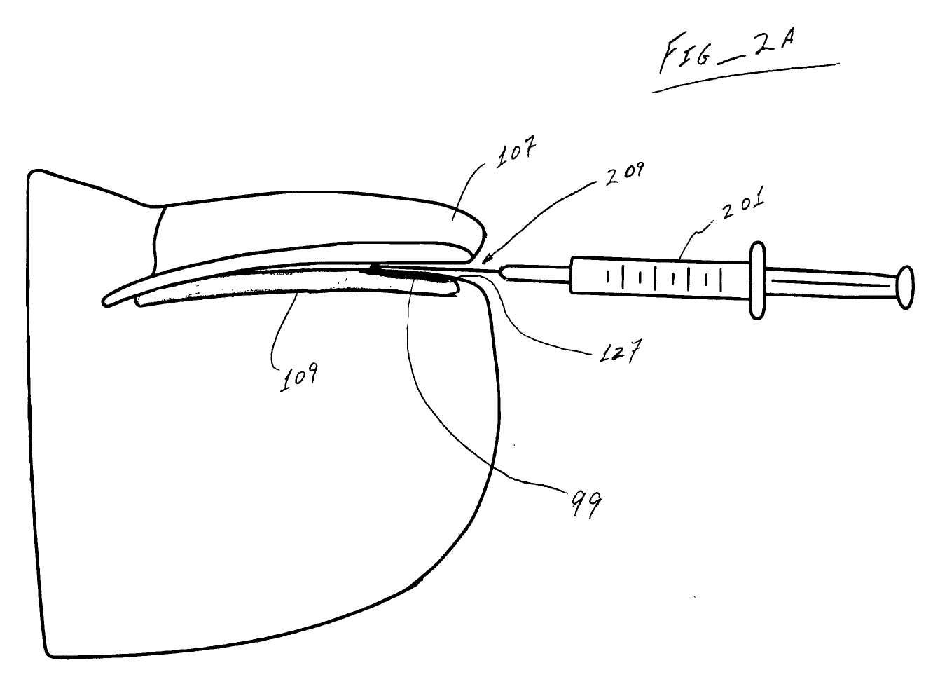

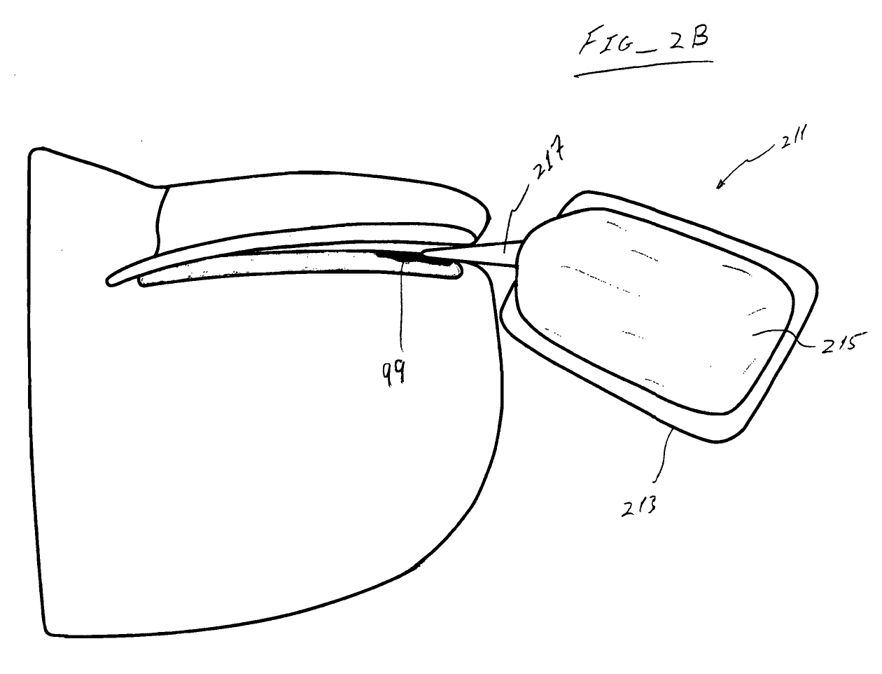

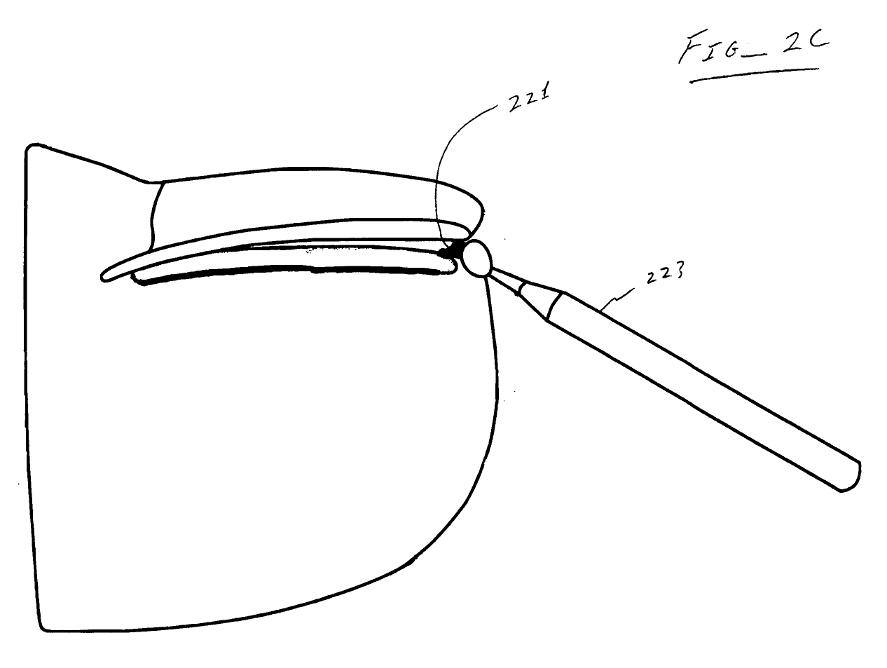

Depending on the individual patient or practitioner's preference, the subunguicide can be administered in the various forms mentioned above. The subunguicide can be administered in a low viscosity flowable form, in which case the device for administering will include a small cannula having sufficient rigidity to be introduced proximal to the hyponychium for delivery of the drug to the nail bed. Such a device can be a hypodermic needle 201 (essentially a cannula having an integral trocar attached to a piston-pressurized reservoir) as shown in FIG. 2A. Also depicted is the effect of onycholysis, the separation of the nail plate from the nail plate starting at the distal end and resulting in a gap 209 between the nail plate and the nail bed that, in a healthy nail, would be sealed by the hyponychium. The relatively low viscosity of such a flowable form is generally less than about 1000 P (100,000 centipoise), and preferably is not dilatant in order to allow use with a piston and hypodermic needle. The administered formulation is shown as 99, and can be colored to provide, even through the nail plate, a visual indication of coverage of the nail bed. FIG. 2B depicts another embodiment of a device for delivering a low or medium viscosity subunquicide. A single-use packet 211 has a relatively rigid backing 213 upon which is formed (such as in the manufacture of blister packs) an overlying flexible wall 215 to provide a reservoir. Alternatively, two flexible walls can be fused or molded together that meet at a seam (215). Formed integrally is cannula 217 for administration of a subunguicide liquid or paste in the reservoir. The end of the cannula is preferably molded to be closed, whereby the user need only cut off the end to use the device. As was shown in FIG. 2A, the cannula is introduced between the hyponychium and the nail plate.

FIG. 2C depicts the subunguicide in the form of a paste 221 forced between the hyponychium and the nail plate with a spatula 223. A paste is used herein to mean a high viscosity fluid, having a viscosity greater than about 1000 P (from the viscosity approximate that of tomato paste or peanut buffer up to that of putty). Beyond that and also suitable for use in this invention are semisolids having effective viscosities over 10,000 P, where the viscosity is typically measured using a penetration test (for example, U.S. Pat. Pub. 20060112503, incorporated herein by reference, for a stick deodorant, appears to use an ASTM test for the viscosity of bituminous materials like asphalt). Administration of a subunguicide in the form of a semisolid (like a deodorant stick) and solids softer than a human nail (like a bar of soap) is shown in FIG. 2D, wherein the patient scratches the surface of the solid or semisolid 231 with sufficient force to propel shavings past the hyponychium and to rest in contact with the nail bed. A solid or semisolid formulation self-administered by scratching has a Mohs hardness less than 2.5 and is sufficiently cohesive that the shavings will be forced past the hyponychium to reside on the nail bed. While this mode of administration may be not advisable if the severity of the onycholysis makes loss of the nail plate possible due to the force required, the softness (viscosity) of the subunguicide in this form can be varied as desired.

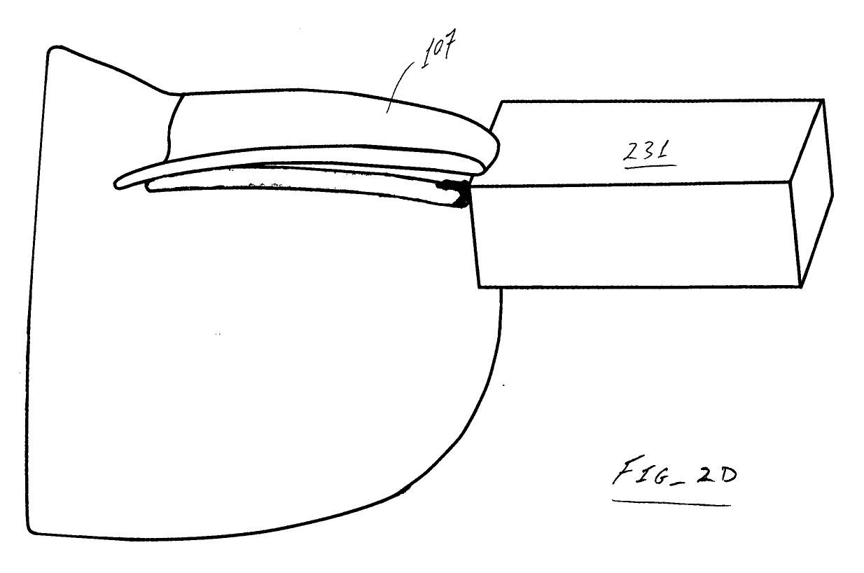

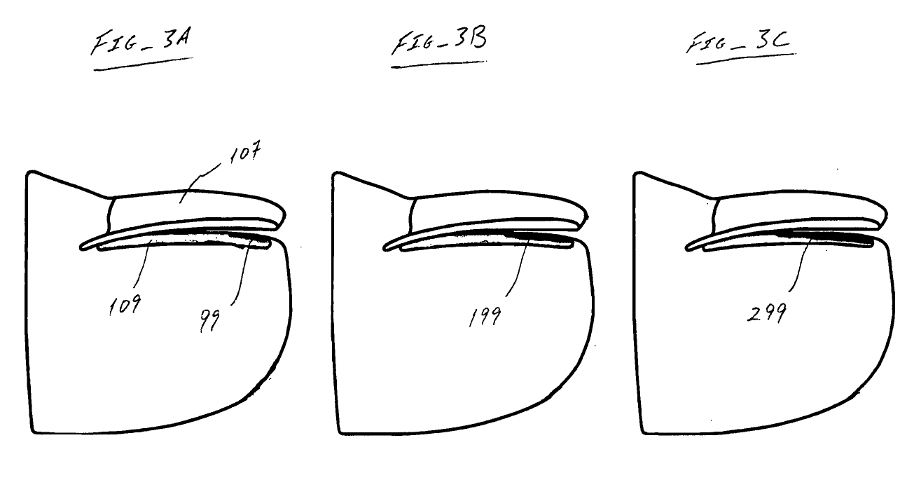

The base used to formulate the subunguicide can be chosen to soften or more preferably liquefy at body temperature (such as a petrolatum) so that the material tends to flow over the nail bed after administration. The chronology shown in FIGS. 3A through 3C depicts migration of the initially applied subunguicide 99 spreading over the nail bed to cover more proximal portions 199 and continuing to cover more proximal and lateral portions 299 of the nail bed. The existence of onycholysis facilitates the migration of the subunguicide proximally.

In yet another embodiment, the subunguicide can be produced in the form of a semisolid or solid disposed in the orifice of a cannula (e.g., hypodermic needle) and after introduction of the cannula inserted as the cannula is retracted to leave behind a dose of the subunguicide.

The foregoing description is meant to be illustrative and not limiting. Various changes, modifications, and additions may become apparent to the skilled artisan upon a perusal of this specification, and such are meant to be within the scope and spirit of the invention as defined by the claims.

What is claimed is:

1. A subunguicide comprising an antifungal agent and a non-soap semisolid carrier having a Mohs hardness less than 2.5 and being sufficiently cohesive when scratched that shavings are propelled past the hyponychium and rest in contact with the nail bed.

2. The subunguicide of claim 1, further comprising a keratolytic agent.

3. The subunguicide of claim 2, wherein the keratolytic agent is an alpha-hydroxy acid, a beta-hydroxy acid, an alpha-keto acid, a beta-keto acid, or a salt thereof.

4. The subunguicide of claim 3, further comprising in combination with the antifungal and keratolytic agent an anti-inflammatory.

5. The subunguicide of claim 1, further comprising an anesthetic.

6. A flowable subunguicide formulation comprising an antifungal agent dispersed in a base exhibiting a viscosity of greater than 1000 P, a hardness less than 2.5 on the Mohs scale.

7. The formulation of claim 6, wherein, the formulation becomes more flowable at or above body temperature.

8. The formulation of claim 6, wherein the viscosity is between 1000 P and 10,000 P.

9. The formulation of claim 6, wherein the viscosity is greater than 10,000 P.

10. The formulation of claim 6, wherein the hardness is between 1 and 2 on the Mohs scale.
